SEQUENCE LISTING (3 sequences)

SEQ ID NO: 1 - DNA, 1134 bp, unidentified
  source 1..1134: mol_type = unassigned DNA
atgtggggaagttcacatgcattcgctggtgaatctgatctgacactacaactacacaccaggtccaacatgagcgacaatacgacaatcaaaaagccgatccgacccaaaccgatccggacggaacgcctgccttacgctggggccgcagaaatcatccgagccaaccagaaagaccactactttgagtccgtgcttgaacagcatctcgtcacgtttctgcagaaatggaagggagtacgatttatccaccagtacaaggaggagctggagacggcgtccaagtttgcatatctcggtttgtgtacgcttgtgggctccaagactctcggagaagagtacaccaatctcatgtacactatcagagaccgaacagctctaccgggggtggtgagacggtttggctacgtgctttccaacactctgtttccatacctgtttgtgcgctacatgggcaagttgcgcgccaaactgatgcgcgagtatccccatctggtggagtacgacgaagatgagcctgtgcccagcccggaaacatggaaggagcgggtcatcaagacgtttgtgaacaagtttgacaagttcacggcgctggaggggtttaccgcgatccacttggcgattttctacgtctacggctcgtactaccagctcagtaagcggatctggggcatgcgttatgtatttggacaccgactggacaagaatgagcctcgaatcggttacgagatgctcggtctgctgattttcgcccggtttgccacgtcatttgtgcagacgggaagagagtacctcggagcgctgctggaaaagagcgtggagaaagaggcaggggagaaggaagatgaaaaggaagcggttgtgccgaaaaagaagtcgtcaattccgttcattgaggatacagaaggggagacggaagacaagatcgatctggaggaccctcgacagctcaagttcattcctgaggcgtccagagcgtgcactctgtgtctgtcatacattagtgcgccggcatgtacgccatgtggacactttttctgttgggactgtatttccgaatgggtgagagagaagcccgagtgtcccttgtgtcggcagggtgtgagagagcagaacttgttgcctatcagataa

SEQ ID NO: 2 - DNA, 1545 bp, unidentified
  source 1..1545: mol_type = unassigned DNA
atgactatcgactcacaatactacaagtcgcgagacaaaaacgacacggcacccaaaatcgcgggaatccgatatgccccgctatcgacaccattactcaaccgatgtgagaccttctctctggtctggcacattttcagcattcccactttcctcacaattttcatgctatgctgcgcaattccactgctctggccatttgtgattgcgtatgtagtgtacgctgttaaagacgactccccgtccaacggaggagtggtcaagcgatactcgcctatttcaagaaacttcttcatctggaagctctttggccgctacttccccataactctgcacaagacggtggatctggagcccacgcacacatactaccctctggacgtccaggagtatcacctgattgctgagagatactggccgcagaacaagtacctccgagcaatcatctccaccatcgagtactttctgcccgccttcatgaaacggtctctttctatcaacgagcaggagcagcctgccgagcgagatcctctcctgtctcccgtttctcccagctctccgggttctcaacctgacaagtggattaaccacgacagcagatatagccgtggagaatcatctggctccaacggccacgcctcgggctccgaacttaacggcaacggcaacaatggcaccactaaccgacgacctttgtcgtccgcctctgctggctccactgcatctgattccacgcttcttaacgggtccctcaactcctacgccaaccagatcattggcgaaaacgacccacagctgtcgcccacaaaactcaagcccactggcagaaaatacatcttcggctaccacccccacggcattatcggcatgggagcctttggtggaattgccaccgagggagctggatggtccaagctctttccgggcatccctgtttctcttatgactctcaccaacaacttccgagtgcctctctacagagagtacctcatgagtctgggagtcgcttctgtctccaagaagtcctgcaaggccctcctcaagcgaaaccagtctatctgcattgtcgttggtggagcacaggaaagtcttctggccagacccggtgtcatggacctggtgctactcaagcgaaagggttttgttcgacttggtatggaggtcggaaatgtcgcccttgttcccatcatggcctttggtgagaacgacctctatgaccaggttagcaacgacaagtcgtccaagctgtaccgattccagcagtttgtcaagaacttccttggattcacccttcctttgatgcatgcccgaggcgtcttcaactacgatgtcggtcttgtcccctacaggcgacccgtcaacattgtggttggttcccccattgacttgccttatctcccacaccccaccgacgaagaagtgtccgaataccacgaccgatacatcgccgagctgcagcgaatctacaacgagcacaaggatgaatatttcatcgattggaccgaggagggcaaaggagccccagagttccgaatgattgagtaa

SEQ ID NO: 3 - DNA, 8468 bp, unidentified
  source 1..8468: mol_type = unassigned DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccgcttttcgtagataatggaatacaaatggatatccagagtatacacatggatagtatacactgacacgacaattctgtatctctttatgttaactactgtgaggcgttaaatagagcttgatatataaaatgttacatttcacagtctgaacttttgcagattacctaatttggtaagatattaattatgaactgaaagttgatggcatccctaaatttgatgaaagggcgcgggatccggcccgggaattcgaaggtaccaaggaagcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggagctcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcggtacccgaattcctgaggtgtctcacaagtgccgtgcagtcccgcccccacttgcttctctttgtgtgtagtgtacgtacattatcgagaccgttgttcccgcccacctcgatccggtctagactgaggtgtctcacaagtgccgtgcagtcccgcccccacttgcttctctttgtgtgtagtgtacgtacattatcgagaccgttgttcccgcccacctcgatccggggatccctgaggtgtctcacaagtgccgtgcagtcccgcccccacttgcttctctttgtgtgtagtgtacgtacattatcgagaccgttgttcccgcccacctcgatccgggtcgacctgaggtgtctcacaagtgccgtgcagtcccgcccccacttgcttctctttgtgtgtagtgtacgtacattatcgagaccgttgttcccgcccacctcgatccgggcatgcctgcagaagcttagagaccgggttggcggcgtatttgtgtcccaaaaaacagccccaattgccccaattgaccccaaattgacccagtagcgggcccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaaatcgttaagcatttccttctgagtataagaatcattcaaaggcgcgccatgactatcgactcacaatactacaagtcgcgagacaaaaacgacacggcacccaaaatcgcgggaatccgatatgccccgctatcgacaccattactcaaccgatgtgagaccttctctctggtctggcacattttcagcattcccactttcctcacaattttcatgctatgctgcgcaattccactgctctggccatttgtgattgcgtatgtagtgtacgctgttaaagacgactccccgtccaacggaggagtggtcaagcgatactcgcctatttcaagaaacttcttcatctggaagctctttggccgctacttccccataactctgcacaagacggtggatctggagcccacgcacacatactaccctctggacgtccaggagtatcacctgattgctgagagatactggccgcagaacaagtacctccgagcaatcatctccaccatcgagtactttctgcccgccttcatgaaacggtctctttctatcaacgagcaggagcagcctgccgagcgagatcctctcctgtctcccgtttctcccagctctccgggttctcaacctgacaagtggattaaccacgacagcagatatagccgtggagaatcatctggctccaacggccacgcctcgggctccgaacttaacggcaacggcaacaatggcaccactaaccgacgacctttgtcgtccgcctctgctggctccactgcatctgattccacgcttcttaacgggtccctcaactcctacgccaaccagatcattggcgaaaacgacccacagctgtcgcccacaaaactcaagcccactggcagaaaatacatcttcggctaccacccccacggcattatcggcatgggagcctttggtggaattgccaccgagggagctggatggtccaagctctttccgggcatccctgtttctcttatgactctcaccaacaacttccgagtgcctctctacagagagtacctcatgagtctgggagtcgcttctgtctccaagaagtcctgcaaggccctcctcaagcgaaaccagtctatctgcattgtcgttggtggagcacaggaaagtcttctggccagacccggtgtcatggacctggtgctactcaagcgaaagggttttgttcgacttggtatggaggtcggaaatgtcgcccttgttcccatcatggcctttggtgagaacgacctctatgaccaggttagcaacgacaagtcgtccaagctgtaccgattccagcagtttgtcaagaacttccttggattcacccttcctttgatgcatgcccgaggcgtcttcaactacgatgtcggtcttgtcccctacaggcgacccgtcaacattgtggttggttcccccattgacttgccttatctcccacaccccaccgacgaagaagtgtccgaataccacgaccgatacatcgccgagctgcagcgaatctacaacgagcacaaggatgaatatttcatcgattggaccgaggagggcaaaggagccccagagttccgaatgattgagtaattaattaaggcacgtgcctaaaaaggatcgataccgtcgacctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcggctaacttgtttaaacaactgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccagatcctctagagtcgaagcggccgctatgtctgataaaaggatgtaacataggcaagctgctcgtgagtgttgagtacgaaccttagatccaaatcacccgcacccacggatatacttgcttgaatatacagtagtatgcggccgcttcgacaccatatcatataaaactaacaatgcatgtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtcatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacaccaaaaccatgatcaaaccacccatggacttcctggaggcagaagaacttgttatggaaaagctcaagagagagaagccaagatactatcaagacatgtgtcgcaacttcaaggaggaccaagctctgtacaccgagaaacaggcctttgtcgacagatctggccctttcgtc

(19) United States
(12) Patent Application Publication
(10) Pub. No.: US 2023/0242948 A1
(43) Pub. Date: Aug. 3, 2023

(54) OLEIC ACID PRODUCED FROM MICROORGANISM AND METHOD FOR PRODUCING OLEIC ACID USING MICROORGANISM

(71) Applicant: KOREA INSTITUTE OF SCIENCE AND TECHNOLOGY, Seoul (KR)

(72) Inventors: Sun Mi LEE, Seoul (KR); Yeon-Ho CHOI, Seoul (KR); Gyeongtaek GONG, Seoul (KR); Ja Kyoung KO, Seoul (KR); Jung Ho AHN, Seoul (KR); Youngsoon UM, Seoul (KR)

(21) Appl. No.: 17/823,034

(22) Filed: Aug. 29, 2022

(30) Foreign Application Priority Data
Jan. 28, 2022 (KR) 10-2022-0013316

(51) Int. Cl. C12P 7/6409; C12N 9/10; C12N 1/16

(52) U.S. Cl. CPC C12P 7/6409; C12N 9/1029; C12Y 203/0102; C12N 1/16

(57) ABSTRACT

The present specification describes a culture of a microorganism, which comprises an increased content of oleic acid, or a microbial oil comprising the same. In addition, the present specification describes a method for producing oleic acid and lipids comprising the same by culturing a microorganism. Since the present disclosure enables the production of lipids comprising oleic acid at a high concentration without genetic manipulation of a lipid-producing microorganism, it may be utilized in various industrial fields requiring oleic acid, such as foods, cosmetic materials, biofuels, etc.

### TECHNICAL FIELD

The present specification discloses a method for producing oleic acid using a microorganism and oleic acid produced by the method.

### CROSS-REFERENCE TO RELATED APPLICATION

This application claims the priority of Korean Patent Application No. 10-2022-0013316, filed on Jan. 28, 2022, the contents of which in their entirety are herein incorporated by reference.

### DESCRIPTION ABOUT NATIONAL SUPPORT RESEARCH AND DEVELOPMENT

This study is conducted by the support of Korea Ministry of Science and ICT under the supervision of Korea Institute of Science and Technology. Research title thereof is Development of medium/long-chain fatty acid production strains and bioprocesses that convert unused biomass (Research management agency: National Research Foundation of Korea, Subject Number: 2020M1A2A2080847, Subject Identification No.: 1711127810).

### REFERENCE TO AN ELECTRONIC SEQUENCE LISTING

The contents of the electronic sequence listing (sequencelisting_457KCL0012US.xml; Size: 14,922 bytes; and Date of Creation: Aug. 24, 2022) is herein incorporated by reference in its entirety.

### BACKGROUND ART

Oleic acid is a major component of vegetable oils. It is used not only as a raw material for biofuels such as biodiesel, aviation biofuel, high-quality lubricating oil, etc., biopolymers, etc. but also as a food or cosmetic material owing to its useful antiaging-related health effects. However, a method of producing oleic acid from vegetable oil has the problems that plants comprising oleic acid are limited, and it is difficult to meet the consistently increasing needs of oleic acid because large cultivation area and long cultivation time are required due to low productivity.

### DISCLOSURE

### Technical Problem

In an aspect, the present disclosure is directed to providing a method for producing oleic acid using a microorganism, which is capable of replacing a method for producing oleic acid from vegetable oil.

The present disclosure is also directed to providing a culture of a microorganism, which comprises oleic acid at an increased content, or a microbial oil comprising lipids isolated therefrom.

### Technical Solution

In an exemplary embodiment, the present disclosure provides a culture of a lipid-producing microorganism, wherein the lipid-producing microorganism is cultured in a medium comprising hexanoic acid, and wherein the culture comprises lipids comprising oleic acid at an increased content as compared to a culture obtained by culturing the lipid-producing strain in a medium not comprising hexanoic acid.

In another exemplary embodiment, the present disclosure provides a microbial oil comprising lipids isolated from the culture of the lipid-producing microorganism.

In another exemplary embodiment, the present disclosure provides a method for producing lipids, which comprises a step of culturing a lipid-producing microorganism in a medium comprising hexanoic acid.

### Advantageous Effects

According to the present disclosure, the production efficiency of lipids comprising oleic acid can be increased effectively by adding hexanoic acid during culturing of a lipid-producing microorganism. The present disclosure can increase the production efficiency by 2000 times or higher per unit area as compared to the existing method of producing from vegetable oil and also can significantly reduce production time. In addition, since the production of oleic acid can be increased simply by adding hexanoic acid during the culturing of the microorganism without transformation through genetic manipulation of the microorganism, the present disclosure is simple, economical and highly sustainable while providing high productivity as compared to the production method using genetic manipulation of a microorganism.

### BRIEF DESCRIPTION OF DRAWINGS

FIG. 1 shows glucose consumption curves for Example 1, Example 2, Comparative Example 1 and Comparative Example 2.

FIG. 2 shows lipid titer and lipid content for Example 1, Example 2, Comparative Example 1 and Comparative Example 2.

FIG. 3 shows oleic acid titer and oleic acid content in lipids for Example 1, Example 2, Comparative Example 1 and Comparative Example 2.

FIG. 4 shows relative oleic acid titer for Example 1, Example 2, Comparative Example 1 and Comparative Example 2.

FIG. 5 shows the composition of lipids produced in Comparative Example 3, Example 3, Example 4 and Example 5.

FIG. 6 shows oleic acid titer for Comparative Example 3, Example 3, Example 4 and Example 5.

FIG. 7 shows glucose consumption curves for Comparative Example 3, Example 3, Example 4 and Example 5.

FIG. 8 shows xylose consumption curves for Comparative Example 3, Example 3, Example 4 and Example 5.

### BEST MODE

The exemplary embodiments disclosed in the present specification are provided only for the purpose of illustrating the present disclosure. The exemplary embodiments of the present disclosure may be carried out in various forms and should not be interpreted as limiting the present disclosure. The present disclosure may be changed variously and may have various forms. Therefore, the exemplary embodiments are not intended to limit the present disclosure but should be understood to include all changes, equivalents and substitutes included in the technical idea and scope of the present disclosure. Singular expressions include plural expressions unless the context clearly indicates otherwise. In this application, terms such as “include”, “have”, etc. are intended to indicate the existence of a feature, material, number, step, element or combinations thereof described in the specification, and should be understood that the existence or possibility of addition of one or more additional features, materials, numbers, steps, elements or combinations is not precluded.

In an exemplary embodiment, the present disclosure may provide a culture of a lipid-producing microorganism, wherein the lipid-producing microorganism is cultured in a medium comprising hexanoic acid, and the culture comprises lipids comprising oleic acid at an increased content as compared to a culture obtained by culturing the lipid-producing strain in a medium not comprising hexanoic acid.

In another exemplary embodiment, the present disclosure may provide a method for producing lipids, which comprises a step of culturing a lipid-producing microorganism in a medium comprising hexanoic acid, wherein a culture obtained by the culturing is the culture of the lipid-producing microorganism described above.

In the present disclosure, the “lipid” refers to an organic substance or an organic compound consisting of a fatty acid and a glycerol. In an exemplary embodiment, the lipid may refer to a microbial oil or an organic substance or an organic compound consisting of a fatty acid and a glycerol accumulated in an organism. In an exemplary embodiment, the lipid may comprise one or more of an acylglycerol, a glyceride and a free fatty acid. Specifically, the acylglycerol may be one or more selected from a group consisting of triacylglycerol (TAG), diacylglycerol (DAG) and monoacylglycerol (MAG). Specifically, the glyceride may be one or more selected from a group consisting of a monoglyceride, a diglyceride and a triglyceride. In an exemplary embodiment, the lipid may be one or more selected from a group consisting of butyric acid (butanoic acid, C4:0), caproic acid (hexanoic acid, C6:0), caprylic acid (octanoic acid, C8:0), capric acid (decanoic acid, C10:0), lauric acid (dodecanoic acid, C12:0), myristic acid (tetradecanoic acid, C14:0), myristoleic acid (ω-5, C14:1), pentadecylic acid (C15:0), palmitic acid (hexadecanoic acid, C16:0), palmitoleic acid (ω-7, C16:1), hexadecadienoic acid (C16:2), hexadecatrienoic acid (C16:3), margaric acid (C17:0), heptadenoic acid (C17:1), stearic acid (octadecanoic acid, C18:0), oleic acid (ω-9, C18:1), linoleic acid (LA, ω-6, C18:2), α-linolenic acid (ALA, ω-3, C18:3), octadecatetraenoic acid (C18:4), nonadecylic acid (C19:0), nonadecylic acid (C19:1), arachidic acid (eicosanoic acid, C20:0), arachidonic acid (AA, ω-6, C20:4), eicosapentaenoic acid (EPA, ω-3, C20:5), behenic acid (docosanoic acid, C22:0), erucic acid (ω-9, C22:1), docosapentanoic acid (DPA, ω-3, 22:5) and docosahexanoic acid (DHA, ω-3, C22:6). Specifically, the lipid may be one or more fatty acid selected from a group consisting of C16:0, C16:1, C16:2, C16:3, C18:0, C18:1, C18:2, C18:3 and C18:4.

In the present disclosure, the “oleic acid” refers to a lipid which is classified as a monosaturated ω-9 fatty acid having 18 carbon atoms and one double bond.

In the present disclosure, the lipid-producing microorganism may be oleaginous yeast that can produce lipids, specifically an oleaginous yeast strain capable of producing lipids. More specifically, the lipid-producing microorganism may be selected from a group consisting of Yarrowia lipolytica, Rhodosporidium toruloides, Rhodotorula glutinis, Trichosporon pullulan, Lipomyces lipofer and Cryptococcus curvatus. However, any microorganism capable of producing lipids may be used without being limited thereto.

In an exemplary embodiment, the lipid-producing microorganism may be a wild-type microorganism or a genetically recombinant microorganism. In an exemplary embodiment, the genetically recombinant microorganism may be one in which a specific gene has been deleted or overexpressed. For example, the genetically recombinant microorganism may be a recombinant microorganism in which one or more of the overexpression of diacylglycerol acyltransferase and the deletion of peroxisome biogenesis factor 10 has been made in the wild-type microorganism, although not being limited thereto. For example, the lipid-producing microorganism may be wild-type Yarrowia lipolytica. In an exemplary embodiment, the wild-type Yarrowia lipolytica may be one which is commercially available or one deposited in a credible agency and can be acquired freely from a catalogue published by the agency. In an exemplary embodiment, the wild-type Yarrowia lipolytica may be a microorganism with the accession number ATCC MYA-2613. But, any wild-type Yarrowia lipolytica may be used without limitation.

For example, the genetically recombinant microorganism may be one in which peroxisomal biogenesis factor 10 (SEQ ID NO 1) has been deleted using the CRISPR/Cas9 system and diacylglycerol acyltransferase has been overexpressed using a vector (pMCS-12TEF-DGA1-CYC1t, SEQ ID NO 3) comprising a UAS1B enhancer, a TEF (translational elongation factor) promoter and a gene encoding diacylglycerol acyltransferase (SEQ ID NO 2) in the wild-type microorganism. In addition, a recombinant strain modified by a general molecular biological technique may also be comprised. In this aspect, the method for producing lipids according to an exemplary embodiment of the present disclosure may further comprise, before the step of culturing the lipid-producing microorganism in a medium comprising hexanoic acid, one or more of a step of overexpressing diacylglycerol acyltransferase in the lipid-producing microorganism and a step of deleting peroxisomal biogenesis factor 10. Specifically, it may comprise a step of deleting peroxisomal biogenesis factor 10 using the CRISPR/Cas9 system and overexpressing diacylglycerol acyltransferase using a vector (pMCS-12TEF-DGA1-CYC1t) comprising a UAS1B enhancer, a TEF (translational elongation factor) promoter and a gene encoding diacylglycerol acyltransferase. In the step of inserting or deleting the genes, the order of the insertion or deletion of the genes is not limited to that described in the present specification.

In the present disclosure, the “culturing” may be conducted by any culturing method known in the art without limitation. In an exemplary embodiment, the microorganism may be cultured by any method selected from a group consisting of shaking culture, stationary culture, batch culture, fed-batch culture and continuous culture. The shaking culture refers to a method of inoculating a microorganism to a medium and culturing the same while shaking, and the stationary culture refers to a method of inoculating a microorganism to a liquid medium and culturing the same without shaking. The batch culture refers to a method of culturing a microorganism with the volume of a medium fixed, i.e., without further adding a fresh medium from outside, and the fed-batch culture, which is contrasted with the batch culture of culturing a microorganism by adding all ingredients to a culture tank at the beginning of the culturing, refers to a method of culturing a microorganism by adding a small amount of ingredients first and then feeding more ingredients during the culturing. The continuous culture refers to a method of culturing a microorganism by continuously supplying a fresh nutrient medium while continuously removing a culture comprising cells and products at the same time.

In an exemplary embodiment, the culturing may be performed in a common medium comprising one or more selected from a group consisting of suitable carbon sources, nitrogen sources, amino acids, vitamins, etc. while controlling culturing conditions such as temperature, pH, etc. For example, the medium may be a YSC (yeast synthetic complete) medium which is a minimal medium. In an exemplary embodiment, the carbon source may comprise a sugar or a carbohydrate such as glucose, xylose, sucrose, lactose, fructose, maltose, starch and cellulose, an oil or a fat such as soybean oil, sunflower oil, castor oil, coconut oil, etc., a fatty acid such as palmitic acid, stearic acid or linoleic acid, an alcohol such as glycerol or ethanol, etc. More specifically, the carbon source may comprise one or more of glucose, glycerol and xylose. These substances may be comprised individually or in admixture. In an exemplary embodiment, the nitrogen source may be an inorganic nitrogen source such as ammonia, ammonium sulfate, ammonium chloride, ammonium acetate, ammonium phosphate, ammonium carbonate and ammonium nitrate; or an organic nitrogen source such as an amino acid, e.g., glutamic acid, methionine and glutamine, peptone, NZ-amine, meat extract, yeast extract, malt extract, corn steep liquor, casein hydrolysate, fish or its degradation product, defatted soybean cake or its degradation product, etc. These nitrogen sources may be used either alone or in combination. The medium may contain monopotassium phosphate, dipotassium phosphate or sodium-containing salts thereof as a phosphorus source. In another exemplary embodiment, dihydrogen potassium phosphate, dipotassium hydrogen phosphate or sodium-containing salts thereof may be used as a phosphorus source. In addition, inorganic compounds such as sodium chloride, calcium chloride, iron chloride, magnesium sulfate, iron sulfate, manganese sulfate, calcium carbonate, etc., may be used. Finally, substances such as amino acids and vitamins may be further used. In an exemplary embodiment, suitable precursors may be comprised in the culture medium. The above-described ingredients may be added to the medium during the culturing in a batch, fed-batch or continuous mode, although not being specially limited thereto. The pH of the culture may be adjusted using a base compound such as sodium hydroxide, potassium hydroxide or ammonia or an acidic compound such as phosphoric acid or sulfuric acid of adequate concentration.

In an exemplary embodiment, the medium may comprise hexanoic acid at a concentration of 0.1-5 g/L. Specifically, the medium may comprise hexanoic acid at a concentration of 0.1 g/L or higher, 0.2 g/L or higher, 0.3 g/L or higher, 0.4 g/L or higher, 0.5 g/L or higher, 0.6 g/L or higher, 0.7 g/L or higher, 0.8 g/L or higher, 0.9 g/L or higher, 1 g/L or higher, 1.1 g/L or higher, 1.5 g/L or higher, 2 g/L or higher, 3 g/L or higher, 4 g/L or higher or 4.9 g/L or higher, and 5 g/L or lower, 4 g/L or lower, 3 g/L or lower, 2 g/L or lower, 1.9 g/L or lower, 1.8 g/L or lower, 1.7 g/L or lower, 1.6 g/L or lower, 1.5 g/L or lower, 1.4 g/L or lower, 1.3 g/L or lower, 1.2 g/L or lower, 1.1 g/L or lower, 1 g/L or lower, 0.9 g/L or lower, 0.8 g/L or lower, 0.7 g/L or lower, 0.6 g/L or lower, 0.5 g/L or lower, 0.4 g/L or lower or 0.3 g/L or lower. If the concentration of the hexanoic acid is too low, the increase in lipid production efficiency may be insignificant. And, if the content of the hexanoic acid exceeds the above ranges, oil productivity may decrease as the growth of the microorganism is inhibited due to the toxicity of the hexanoic acid.

In an exemplary embodiment, in the culturing step, a lipid-producing microorganism may be inoculated to the medium and then the microorganism may be cultured at predetermined temperature and time ranges. For example, the microorganism may be cultured at 24-35° C. Specifically, the culturing temperature may be 24° C. or higher, 25° C. or higher, 26° C. or higher, 27° C. or higher, 28° C. or higher, 29° C. or higher, 30° C. or higher, 31° C. or higher, 32° C. or higher, 33° C. or higher or 34° C. or higher, and 35° C. or lower, 34° C. or lower, 33° C. or lower, 32° C. or lower, 31° C. or lower, 30° C. or lower, 29° C. or lower, 28° C. or lower, 27° C. or lower, 26° C. or lower or 25° C. or lower. For example, the microorganism may be cultured for 24-300 hours. Specifically, the culturing time may be 24 hours or longer, 30 hours or longer, 40 hours or longer, 50 hours or longer, 60 hours or longer, 70 hours or longer, 80 hours or longer, 90 hours or longer, 100 hours or longer, 110 hours or longer, 120 hours or longer, 130 hours or longer, 140 hours or longer, 150 hours or longer, 160 hours or longer, 170 hours or longer, 180 hours or longer, 190 hours or longer, 200 hours or longer, 210 hours or longer, 220 hours or longer, 230 hours or longer, 240 hours or longer, 250 hours or longer, 260 hours or longer, 270 hours or longer, 280 hours or longer or 290 hours or longer, and 300 hours or shorter, 290 hours or shorter, 280 hours or shorter, 270 hours or shorter, 260 hours or shorter, 250 hours or shorter, 240 hours or shorter, 230 hours or shorter, 220 hours or shorter, 210 hours or shorter, 200 hours or shorter, 190 hours or shorter, 180 hours or shorter, 170 hours or shorter, 160 hours or shorter, 150 hours or shorter, 140 hours or shorter, 130 hours or shorter, 120 hours or shorter, 110 hours or shorter, 100 hours or shorter, 90 hours or shorter, 80 hours or shorter, 70 hours or shorter, 60 hours or shorter, 50 hours or shorter, 40 hours or shorter, 30 hours or shorter or 25 hours or shorter. If the culturing temperature or culturing time is outside the above ranges, the growth and composition of the microorganism may be affected and, thus, oil productivity may be decreased.

In an exemplary embodiment, the culture of the microorganism may have an oleic acid content increased by 10-80%.

In an exemplary embodiment, the content of lipids other than oleic acid in the culture of the microorganism may be increased by 5-30%.

In addition, in an exemplary embodiment of the present disclosure, the utilization of various carbon sources other than glucose, such as xylose, by the microorganism may be increased by adding hexanoic acid to the culture medium of the microorganism. In addition, in an exemplary embodiment, the speed of production of lipids comprising oleic acid by the microorganism may be increased by adding hexanoic acid to the culture medium of the microorganism.

In an exemplary embodiment, the present disclosure may provide a microbial oil comprising lipids isolated from a culture of a lipid-producing microorganism obtained by culturing the same in a medium comprising hexanoic acid. In addition, in an exemplary embodiment, the present disclosure may provide a food, a cosmetic, a biofuel, a bioplastic material, etc. comprising the lipids or the microbial oil. For example, in an exemplary embodiment, the produced oleic acid may be converted to ω3, DHA, EPA, etc. through chain elongation and unsaturation and a functional health food comprising the same may be provided. In addition, in an exemplary embodiment, a method for producing microbial oil, which comprises a step of producing lipid according to the method for producing lipids described above, may be provided. Specifically, a method for producing microbial oil using the culture of the strain described above may be provided. For example, the method may further comprise a step of preparing a biodiesel by transesterifying the produced lipids. For example, the method may further comprise a step of preparing an aviation biofuel by hydrocracking the produced lipids.

Hereinafter, the present disclosure is described more specifically referring to examples and drawings. However, the following examples and drawings are provided only to facilitate the understanding of the present disclosure and the scope of the present disclosure is not limited by them.

### EXAMPLE 1

As an exemplary embodiment of the present disclosure, wild-type Yarrowia lipolytica (microorganism accession number ATCC MYA-2613) was inoculated to a minimal medium containing glucose as a carbon source (20 g/L glucose, 6.7 g/L yeast nitrogen base, CSM-ura (MP Biomedicals, Solon, USA), pH 6.8 potassium phosphate buffer) and supplemented with 1 g/L hexanoic acid. Then shaking culture was conducted at 28° C. and 200 rpm. After culturing for 144-168 hours, the microorganism was separated from the culture through centrifugation.

### EXAMPLE 2

As an exemplary embodiment of the present disclosure, after deleting peroxisomal biogenesis factor 10 (SEQ ID NO 1), which is a lipid degradation-related gene, from the wild-type Yarrowia lipolytica (microorganism accession number ATCC MYA-2613) of Example 1 using the CRISPR/Cas9 system, a lipid-producing microorganism was prepared by overexpressing diacylglycerol acyltransferase, which is a key gene in a lipid-producing metabolic pathway, using a vector (pMCS-12TEF-DGA1-CYC1t, SEQ ID NO 3) comprising a UAS1B enhancer, a TEF (translational elongation factor) promoter and a gene encoding diacylglycerol acyltransferase (SEQ ID NO 2). Then, after inoculating the microorganism to a minimal medium containing glucose as a carbon source (20 g/L glucose, 6.7 g/L yeast nitrogen base, CSM-ura (MP Biomedicals, Solon, USA), pH 6.8 potassium phosphate buffer) and supplemented with 1 g/L hexanoic acid, shaking culture was conducted at 28° C. and 200 rpm. After culturing for 144-168 hours, the microorganism was separated from the culture through centrifugation.

### Comparative Example 1

After inoculating wild-type Yarrowia lipolytica (microorganism accession number ATCC MYA-2613) to a minimal medium containing glucose as a carbon source (20 g/L glucose, 6.7 g/L yeast nitrogen base, CSM-ura (MP Biomedicals, Solon, USA), pH 6.8 potassium phosphate buffer), shaking culture was conducted at 28° C. and 200 rpm. After culturing for 144-168 hours, the microorganism was separated from the culture through centrifugation.

### Comparative Example 2

After deleting peroxisomal biogenesis factor 10 (SEQ ID NO 1), which is a lipid degradation-related gene, from the wild-type Yarrowia lipolytica (microorganism accession number ATCC MYA-2613) of Example 1 using the CRISPR/Cas9 system, a lipid-producing microorganism was prepared by overexpressing diacylglycerol acyltransferase, which is a key gene in a lipid-producing metabolic pathway, using a vector (pMCS-12TEF-DGA1-CYC1t, SEQ ID NO 3) comprising a UAS1B enhancer, a TEF (translational elongation factor) promoter and a gene encoding diacylglycerol acyltransferase (SEQ ID NO 2). Then, after inoculating the microorganism to a minimal medium containing glucose as a carbon source (20 g/L glucose, 6.7 g/L yeast nitrogen base, CSM-ura (MP Biomedicals, Solon, USA), pH 6.8 potassium phosphate buffer), shaking culture was conducted at 28° C. and 200 rpm. After culturing for 144-168 hours, the microorganism was separated from the culture through centrifugation.

### Test Example 1

In order to investigate the consumption of the carbon source in the medium and the content of the produced lipids depending on the addition of hexanoic acid during the culturing of the lipid-producing microorganisms, the amount of biomass (dry cell weight) and lipids produced from the microorganisms isolated in Examples 1-2 and Comparative Examples 1-2 was measured. The amount of produced oleic acid was calculated through lipid assay and the amount of consumed glucose was measured form the separated cultures.

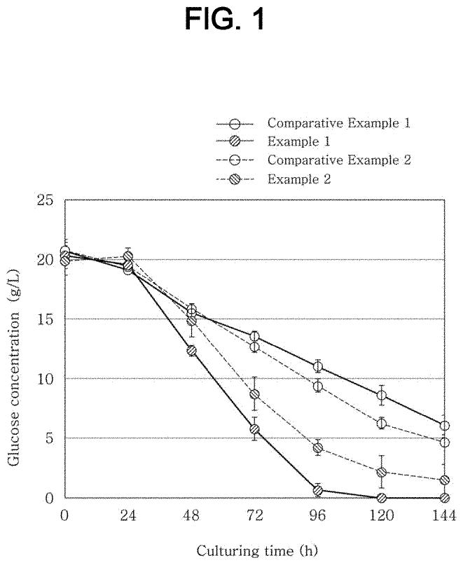

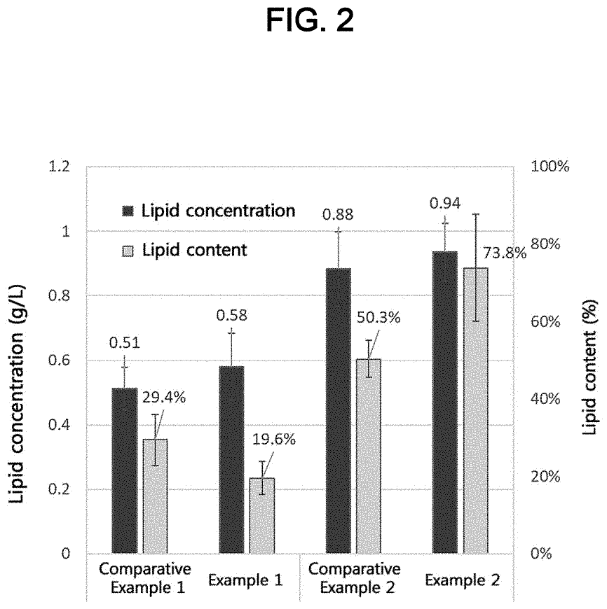

As shown in FIG. 1, the consumption rate of the carbon source glucose was increased by the addition of hexanoic acid for both the lipid-producing microorganisms of Examples 1 and 2. And, as shown in FIG. 2, lipid production was increased by the addition of hexanoic acid by about 10% as compared to Comparative Examples 1 and 2. The lipid production was increased by about 1.7 times for Example 2 as compared to Example 1. For Example 2, the intracellular lipid content was increased from 50.3% to 73.8% by the addition of hexanoic acid as compared to Comparative Example 2. Accordingly, it was confirmed that the improvement in lipid productivity by the addition of hexanoic acid was greater for the recombinant microorganism.

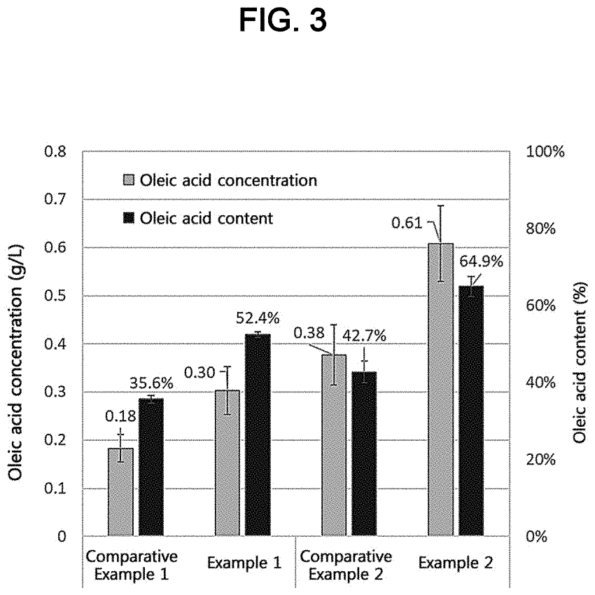

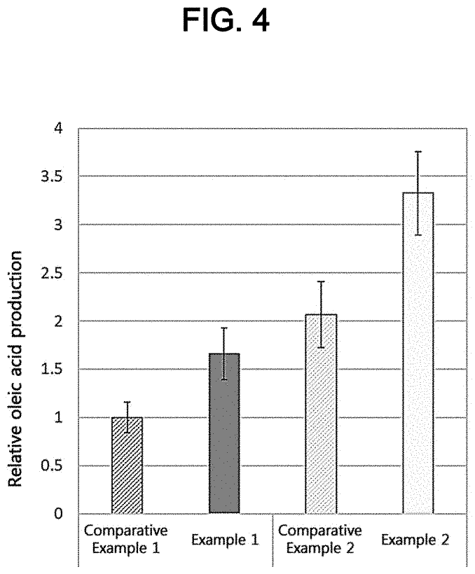

In addition, as a result of analyzing the contents of lipids and oleic acid produced in Examples 1-2 and Comparative Examples 1-2 and comparing the oleic acid production depending on the addition of hexanoic acid therefrom, the addition of hexanoic acid significantly improved the productivity of oleic acid, differently from the productivity of lipids, as shown in FIG. 3. For Example 1 wherein the wild-type lipid-producing microorganism was used, the lipid production was not increased significantly by the addition of hexanoic acid, but the content of oleic acid in the produced lipids was increased by 47% to 66%. Also, for Example 2, the addition of hexanoic acid increased the content of oleic acid in the produced lipids by about 52% to 61%. Accordingly, it was confirmed that about 3.3 times of oleic acid could be produced for Example 2 as compared to Comparative Example 1 wherein the wild-type strain was cultured in a medium not comprising hexanoic acid, as shown in FIG. 4.

### Test Example 2

Experiment was conducted as follows to observe the change in the production of oleic acid depending on the concentration of added hexanoic acid.

Specifically, wild-type Yarrowia lipolytica was transformed in the same manner as in Example 2 and hexanoic acid at 0 g/L (Comparative Example 3), 0.2 g/L (Example 3), 0.6 g/L (Example 4) or 1 g/L (Example 5) was added to a minimal medium containing 20 g/L glucose and 10 g/L xylose as carbon sources (20 g/L glucose, 10 g/L xylose, 6.7 g/L yeast nitrogen base, CSM-ura (MP Biomedicals, Solon, USA), pH 6.8 potassium phosphate buffer). Then, after inoculating the transformed microorganism to the medium, shaking culture was conducted at 28° C. and 200 rpm. After culturing for 168 hours, the microorganism was separated from the culture through centrifugation and the amount of produced lipids was measured from the separated strain. The amount of produced oleic acid was calculated through lipid assay and the amount of glucose and xylose consumed as carbon sources was measured from the separated culture.

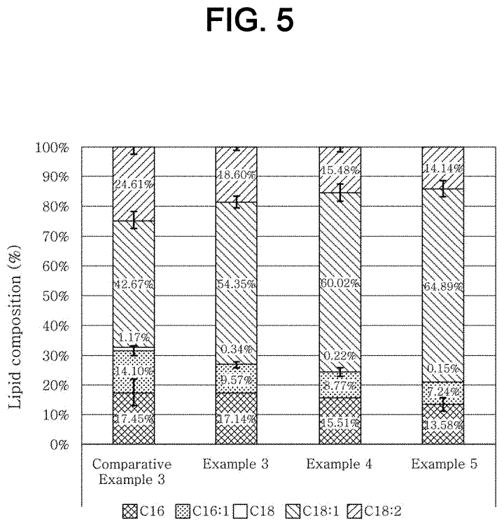

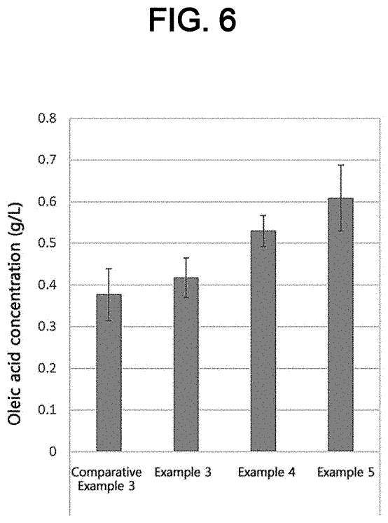

As shown in FIG. 5 and FIG. 6, the oleic acid production was increased as the concentration of hexanoic acid added to the medium was increased. Specifically, when compared with the oleic acid concentration 0.377 g/L of Comparative Example 3 wherein hexanoic acid was not added to the medium, the oleic acid content was increased by 27.4% and the oleic acid concentration was increased to 0.417 g/L by 10.5% for Example 3 wherein 0.2 g/L hexanoic acid was added to the medium, the oleic acid content was increased by 40.1% and the oleic acid concentration was increased to 0.529 g/L by 40.3% for Example 4 wherein 0.6 g/L hexanoic acid was added to the medium, and the oleic acid content was increased by 52.1% and the oleic acid concentration was increased to 0.608 g/L by 61.2% for Example 5 wherein 1 g/L hexanoic acid was added to the medium.

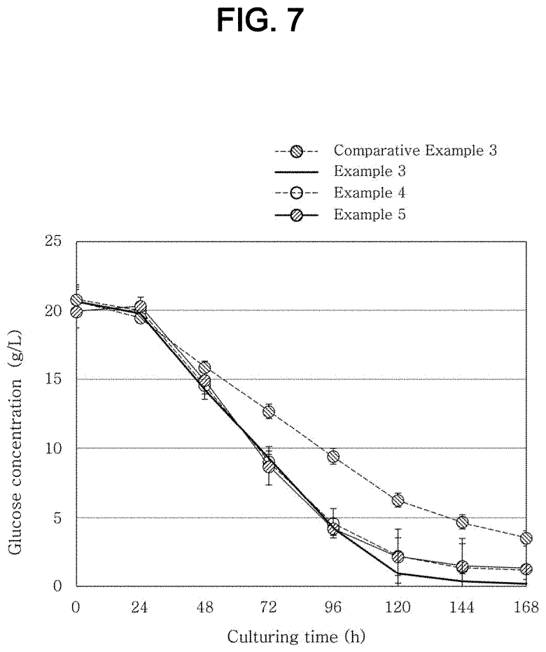

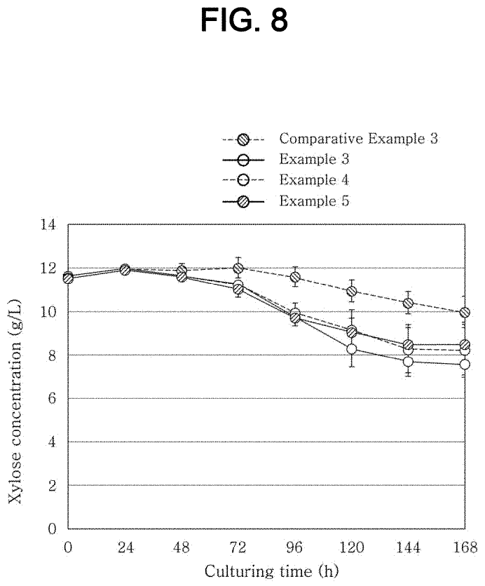

Also, as shown in FIG. 7 and FIG. 8, it was confirmed that, although the Yarrowia lipolytica strain hardly utilized xylose, the consumption of xylose by the Yarrowia lipolytica strain was increased remarkably as hexanoic acid was added to the medium. This means that the addition of hexanoic acid allows the utilization of not only glucose but also various carbon sources such as xylose and can increase the production amount and rate of oleic acid and lipids by facilitating the utilization.

The present disclosure may provide the following exemplary embodiments.

A first exemplary embodiment may provide a culture of a lipid-producing strain, wherein the lipid-producing strain is cultured in a medium comprising hexanoic acid, and wherein the culture comprises lipids comprising oleic acid at an increased content as compared to a culture obtained by culturing the lipid-producing strain in a medium not comprising hexanoic acid.

A second exemplary embodiment may provide the culture according to the first exemplary embodiment, wherein the lipid-producing strain is selected from a group consisting of Yarrowia lipolytica, Rhodosporidium toruloides, Rhodotorula glutinis and Cryptococcus curvatus.

A third exemplary embodiment may provide the culture according to the first or second exemplary embodiment, wherein the lipid-producing strain is a recombinant strain in which one or more of the overexpression of diacylglycerol acyltransferase and the deletion of peroxisome biogenesis factor 10 has been made in a wild-type microorganism.

A fourth exemplary embodiment may provide the culture according to any one of the first to third exemplary embodiments, wherein the medium comprises hexanoic acid at a concentration of 0.1-5 g/L.

A fifth exemplary embodiment may provide a microbial oil comprising lipids isolated from the culture according to the first to fourth exemplary embodiments.

A sixth exemplary embodiment may provide a biofuel comprising lipids isolated from the culture according to any one of the first to fourth exemplary embodiments.

A seventh exemplary embodiment may provide a cosmetic comprising lipids isolated from the culture according to any one of the first to fourth exemplary embodiments.

An eighth exemplary embodiment may provide a food comprising lipids isolated from the culture according to any one of the first to fourth exemplary embodiments.

A ninth exemplary embodiment may provide a method for producing lipids, which comprises a step of culturing a lipid-producing strain in a medium comprising hexanoic acid, whereby the culture according to any one of the first to fourth exemplary embodiments is obtained.

A tenth exemplary embodiment may provide the method for producing lipids according to the ninth exemplary embodiment, wherein the medium comprises one or more of glucose, glycerol and xylose as carbon sources.

An eleventh exemplary embodiment may provide the method for producing lipids according to the ninth or tenth exemplary embodiment, which comprises a step of inoculating a lipid-producing strain to the medium and then culturing the same at 24-35° C. for 24-300 hours.

A twelfth exemplary embodiment may provide a method for producing a microbial oil, which comprises a step of producing lipids by the method for producing lipids according to any one of the ninth to eleventh exemplary embodiments.

A thirteenth exemplary embodiment may provide the method for producing a microbial oil according to the twelfth exemplary embodiment, which further comprises a step of preparing a biodiesel by transesterifying the produced lipids.

A fourteenth exemplary embodiment may provide the method for producing a microbial oil according to the twelfth or thirteenth exemplary embodiment, which further comprises a step of preparing an aviation biofuel by hydrocracking the produced lipids.

1. A culture of a lipid-producing strain, wherein the lipid-producing strain is cultured in a medium comprising hexanoic acid, and
wherein the culture comprises lipids comprising oleic acid at an increased content as compared to a culture obtained by culturing the lipid-producing strain in a medium not comprising hexanoic acid.

2. The culture according to claim 1, wherein the lipid-producing strain is selected from a group consisting of Yarrowia lipolytica, Rhodosporidium toruloides, Rhodotorula glutinis and Cryptococcus curvatus.

3. The culture according to claim 1, wherein the lipid-producing strain is a recombinant strain in which one or more of the overexpression of diacylglycerol acyltransferase and the deletion of peroxisome biogenesis factor 10 has been made in a wild-type microorganism.

4. The culture according to claim 1, wherein the medium comprises hexanoic acid at a concentration of 0.1-5 g/L.

5. A microbial oil comprising lipids isolated from the culture according to claim 1.

6. The microbial oil according to claim 5, wherein the culture is a culture of a lipid-producing strain selected from a group consisting of Yarrowia lipolytica, Rhodosporidium toruloides, Rhodotorula glutinis and Cryptococcus curvatus.

7. The microbial oil according to claim 5, wherein the culture is a culture of a lipid-producing strain which is a recombinant strain in which one or more of the overexpression of diacylglycerol acyltransferase and the deletion of peroxisome biogenesis factor 10 has been made in a wild-type microorganism.

8. The microbial oil according to claim 5, wherein the culture is obtained by culturing the lipid-producing strain in a medium comprising hexanoic acid at a concentration of 0.1-5 g/L.

9. A biofuel comprising lipids isolated from the culture according to claim 1.

10. The biofuel according to claim 9, wherein the culture is a culture of a lipid-producing strain selected from a group consisting of Yarrowia lipolytica, Rhodosporidium toruloides, Rhodotorula glutinis and Cryptococcus curvatus.

11. The biofuel according to claim 9, wherein the culture is a culture of a lipid-producing strain which is a recombinant strain in which one or more of the overexpression of diacylglycerol acyltransferase and the deletion of peroxisome biogenesis factor 10 has been made in a wild-type microorganism.

12. The biofuel according to claim 9, wherein the culture is obtained by culturing the lipid-producing strain in a medium comprising hexanoic acid at a concentration of 0.1-5 g/L.

13. A cosmetic comprising lipids isolated from the culture according to claim 1.

14. The cosmetic according to claim 13, wherein the culture is a culture of a lipid-producing strain selected from a group consisting of Yarrowia lipolytica, Rhodosporidium toruloides, Rhodotorula glutinis and Cryptococcus curvatus.

15. The cosmetic according to claim 13, wherein the culture is a culture of a lipid-producing strain which is a recombinant strain in which one or more of the overexpression of diacylglycerol acyltransferase and the deletion of peroxisome biogenesis factor 10 has been made in a wild-type microorganism.

16. The cosmetic according to claim 13, wherein the culture is obtained by culturing the lipid-producing strain in a medium comprising hexanoic acid at a concentration of 0.1-5 g/L.

17. A food comprising lipids isolated from the culture according to claim 1.

18. The food according to claim 17, wherein the culture is a culture of a lipid-producing strain selected from a group consisting of Yarrowia lipolytica, Rhodosporidium toruloides, Rhodotorula glutinis and Cryptococcus curvatus.

19. The food according to claim 17, wherein the culture is a culture of a lipid-producing strain which is a recombinant strain in which one or more of the overexpression of diacylglycerol acyltransferase and the deletion of peroxisome biogenesis factor 10 has been made in a wild-type microorganism.

20. The food according to claim 17, wherein the culture is obtained by culturing the lipid-producing strain in a medium comprising hexanoic acid at a concentration of 0.1-5 g/L.
